(19) United States
(12) Patent Application Publication
(10) Pub. No.: US 2019/0104776 A1
(43) Pub. Date: Apr. 11, 2019

(54) SYSTEM FOR REGULATING BODY TEMPERATURE OF A SUBJECT

(71) Applicant: Janatec Technologies, Sudbury (CA)

(72) Inventors: Steffon Jon Luoma, Sudbury (CA); Wendell Jason Buie, Little Current (CA); Stephen Alexis Podrucky, Garson (CA)

(21) Appl. No.: 16/065,060

(22) PCT Filed: Dec. 22, 2016
(86) PCT No.: PCT/CA2016/051523

§ 371 Date: Jun. 21, 2018

(30) Foreign Application Priority Data
Dec. 22, 2015 (CA) 2,916,131

(51) Int. Cl. A41D 13/005; A41D 1/00; A41D 31/00; A41D 1/04; A41D 3/00; A41D 1/06; A41D 13/02; A42B 1/00; A42B 3/28; B32B 15/20; B32B 27/06; A61F 7/02; A61B 5/0205; A61B 5/00

(52) U.S. Cl. CPC A41D 13/0051; A61F 2007/0075; A41D 13/0053; A41D 31/0038; A41D 1/04; A41D 3/00; A41D 1/06; A41D 13/02; A42B 1/008; A42B 3/285; A41D 31/0016; B32B 15/20; B32B 27/06; A61F 7/02; A61B 5/02055; A61B 5/6805; A61B 5/6803; A61B 5/4836; A41D 2400/12; B32B 2307/7265; B32B 2307/304; B32B 2307/206; B32B 2437/00; A41D 1/002

(57) ABSTRACT

Disclosed is a system for regulating body temperature of a subject. The system includes: one or more sensors for monitoring environmental and/or physiological parameters; a multi-layered garment having a plurality of thermoelectric elements distributed throughout the multi-layered garment; a controller for receiving input from the one or more sensors and connected to the plurality of thermoelectric elements to systemically control the thermoelectric elements based on the environmental and/physiological parameters; and a battery for providing power to the one or more sensors, the thermoelectric elements, the controller or a combination of any of these. The system can improve worker safety and comfort in an energy efficient manner.

### FIELD OF THE INVENTION

Generally, the present invention is directed to thermoregulating garments. More specifically, the invention is directed to a system for regulating body temperature of a subject based on environmental and/or physiological parameters.

### BACKGROUND OF THE INVENTION

Over the years, more attention has been paid to worker safety, not just from the perspective of hazards in the workplace, but also from the short- and long-term physical effects of working in an environment where the conditions can be extreme, such as in deep underground mines and in forest firefighting situations.

Taking deep underground mines as an example, the ambient temperature of the mine can be consistently above 30° C. and in excess of 60% humidity. In this environment it is recommended that workers follow a work-rest regiment as outlined in the American conference of Governmental Industrial Hygienists (ACGIH) guidelines. Such guidelines recommend the Threshold Limit Values (TLV) for workers and may results in work being performed for 15 minutes followed by a 45 minute resting period, or even indicate that no work should be performed due to heat. This type of efficiency can cause significant strain on the profitability of a mine. Moreover, the extreme conditions can result in workers removing safety equipment in an effort to cool down.

Attempts have been made to provide clothing that is capable of being cooled. However, most of these garments provide a cooling function by the user manually turning on the cooling system and turning it off, once a desired comfort level is achieved. Moreover, the cooling effect in these garments is experienced throughout the garment, which can unnecessarily cool muscles, organs and tissues that are not under heat stress. Since these garments are in an “all on” state, when in operation, the power requirements to keep the system functioning are higher than what would be expected from a system that is selectively turned on when needed. As such, additional or larger power sources must be carried by the user, which can lead to further discomfort and muscle strain.

Based on the foregoing, there is a need for a garment that is capable of automatically regulating a user's body temperature in a selectively and systematic manner, and which optimizes the power required to operate.

### SUMMARY OF THE INVENTION

According to an aspect of the present invention, there is provided a system for regulating body temperature of a subject. The system includes: one or more sensors for monitoring environmental and/or physiological parameters; a multi-layered garment comprising a plurality of thermoelectric elements distributed throughout the multi-layered garment; a controller for receiving input from the one or more sensors and connected to the plurality of thermoelectric elements to systemically control the thermoelectric elements based on the environmental and/physiological parameters; and a battery for providing power to the one or more sensors, the thermoelectric elements, the controller or a combination of any of these.

In one embodiment, the environmental parameters are ambient temperature, humidity, barometric pressure, air velocity or any combination of these and/or the physiological parameters are body temperature, heart rate, heart rate variability, blood pressure, activity level, breathing rate, muscle activity, skin temperature, heat flux or a combination of these.

In another embodiment, the multi-layered garment comprises an outer layer, a heat-sinking layer, an insulating layer, a cooling layer and an inner layer. The outer layer provides protection from the elements and is, optionally, thermally conductive and electrically insulated and is, optionally, waterproof. The insulating layer is electrically and thermally insulated. The inner layer is thermally conductive and electrically insulated and can be capable of transferring heat away from the subject. The thermoelectric elements transverse the heat-sinking, insulating and cooling layers.

In a further embodiment, the multi-layered garment comprises an outer layer, a heat-sinking layer, an insulating layer, a cooling or heating layer and an inner layer. The outer layer provides protection from the elements and is, optionally, thermally conductive and electrically insulated and is, optionally, waterproof. The insulating layer is electrically and thermally insulated. The inner layer is thermally conductive and electrically insulated and can be capable of transferring heat away from the subject.

In a still further embodiment, the thermoelectric elements are controlled by varying the current and/or voltage supplied to the thermoelectric elements.

In a yet further embodiment, the garment is a vest, jacket, trousers, jumpsuit, hat, helmet, or any combination of these.

In one embodiment, the system further comprises fans for cooling the heat-sinking and/or outer layers.

In a further embodiment, the battery is mounted on the garment.

In a still further embodiment, one of the sensors is for mounting intraaurally and/or on the skin of the subject to measure body temperature and heart rate.

In a yet further embodiment, the battery is a portable battery and one of the sensors is a non-invasive physiological sensor.

According to another aspect of the present invention, there is provided a method for regulating body temperature of a subject. The method comprising the steps of: obtaining environmental and/or physiological parameters; processing said environment and/or physiological parameters in a controller; and systematically controlling a plurality of thermoelectric elements distributed throughout a multi-layer garment worn by the subject based on said environmental and/or physiological parameters.

### BRIEF DESCRIPTION OF THE DRAWINGS

These and other features, aspects and advantages of the present invention will become better understood with regard to the following description and accompanying drawings wherein:

FIG. 1 is an illustration of the system according to an embodiment of the present invention;

FIG. 2 is an illustration of the sensors and controller according to an embodiment of the present invention; and

FIG. 3 is a cross-section of the multi-layered garment according to an embodiment of the present invention.

### DESCRIPTION OF THE INVENTION

The following description is of one particular embodiment by way of example only and without limitation to the combination necessary for carrying the invention into effect.

A system for regulating body temperature of a subject is provided. The system monitors environmental and/or physiological parameters and automatically adjusts the temperature of a garment worn by the subject to counteract the environmental and/or physiological parameters or stressors. For example, in a high temperature environment the system would systematically activate cooling features in the vest of a jacket or jumpsuit worn by the subject to cool the body, thus limiting the possibility of heat exhaustion.

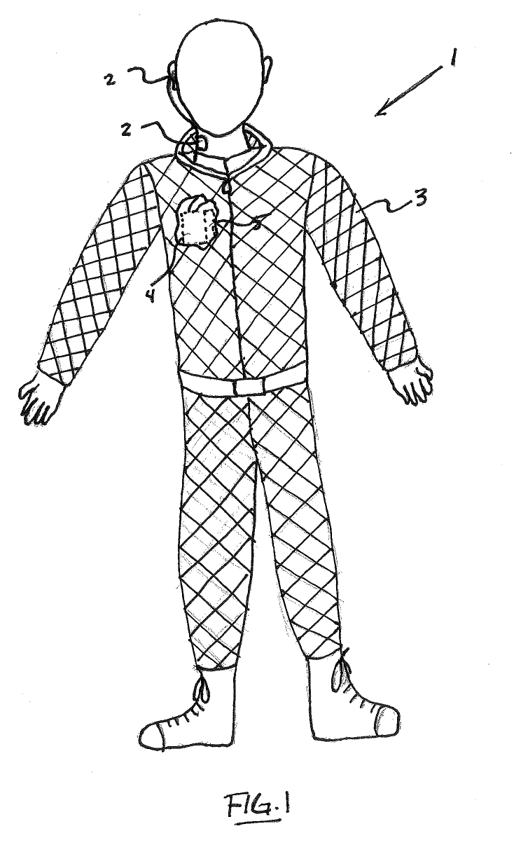

As shown in FIG. 1, the system (1) includes: one or more sensors (2); a multi-layered garment (3); a controller (4); and a battery (5) for providing power to the system (1). In the embodiment shown in FIG. 1, the multi-layered garment (5) is a hooded jumpsuit. However, the multi-layered garment can take many forms, such as, but not limited to, vests, jackets, trousers, hats, helmets, or any combination of these. In the context of the present invention, a garment is any article of clothing that can be worn by a subject, and which can accommodate a plurality of thermoelectric elements, as described below.

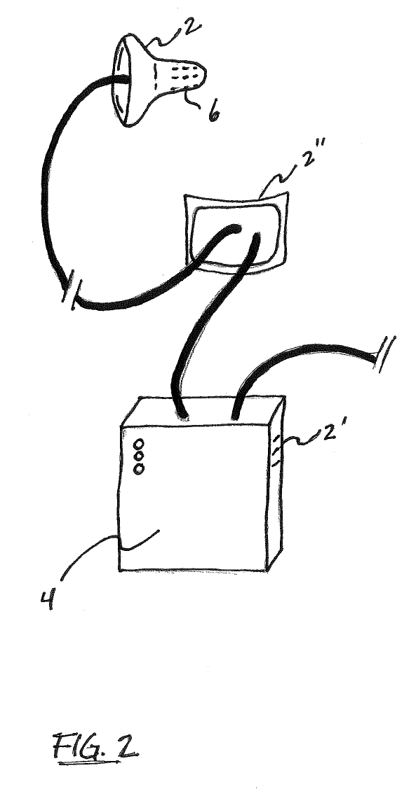

As shown in FIG. 2, the system (1) includes one or more sensors (2) for monitoring environmental and/or physiological parameters. Examples of such environmental parameters include, but are not limited to: ambient temperature; relative humidity; barometric pressure; and air velocity, whereas, examples of physiological parameters include: skin temperature; heart rate; heart rate variability; blood pressure; activity level; breathing rate; muscle activity; body temperature; and heat flux. These sensors (2) can be provided within a single unit, or as separate units that each monitor a separate parameter or a series of parameters belonging to a single category, such as environmental or physiological parameters. In most cases, several sensors will be provided, these sensors will be used to monitor environmental parameters (2) and to monitor physiological parameters (2′). However, additional sensor(s) (2″) can be provided to monitor other environmental or physiological parameters, such as an accelerometer to measure activity, or to provide secondary (or redundant) readings of the environmental or physiological parameters. In one non-limiting example, an intraaural sensor (2) is provided to monitor internal body temperature and/or heart rate of the user. Optionally, the intraaural sensor (2) can be provided with a speaker and/or microphone, such as a bone microphone, to allow for information to be communicated to and from the user. The intraaural sensor (2) is either connected directly to the controller (4), or can be connected to an additional sensor or data bus (2″) that can, as illustrated in the embodiment shown in FIG. 2, be attached to or in the vicinity of the skin of the user, such as on the forehead or within a hard hat suspension (or on the headband) to act as a secondary monitor of the user's body temperature and/or heart rate. Instead of using an intraaural sensor, it is also possible to use only a sensor positioned on or in the vicinity of the skin to measure key physiological parameters, such as skin temperature and heart rate. For example, photoplethysmogram (PPG) sensors can be used to measure the heart rate of the user. The connection between the various sensors and/or controller can be wired or wireless. The additional sensor (2″) can also monitor additional parameters that may not be able to be detected using an intraaural sensor (2), such as, skin temperature, skin heat flux, breathing rate and/or muscle activity. The actual form for such intraaural and skin mounted temperature and heart rate sensors (2, 2″) will be known to those skilled in the art. As shown in FIG. 2, the skin mounted (2″) sensor is connected to a controller unit (4). Additional sensors can be positioned on the user or the garment to detect or monitor additional environmental and/physiological parameters. As with the sensors (2, 2″) shown in the FIG. 2, these additional sensors can be connected directly to the controller unit (4), or can be connected in series with one or more other sensors.

As shown in FIG. 2, the controller unit (4) can have sensors (2′) built therein, or can be a standalone unit that is either positioned in the vicinity or on the user, or can be hosted remotely. In the illustrated embodiment, sensor (2′) is provided to monitor environmental parameters, such as, but not limited to, ambient temperature, humidity, barometric pressure, air velocity or any combination of these.

The data received by the sensors is relayed to the controller unit (4), where the data is transformed using an algorithm that determines the physiological strain index of the user. The algorithm uses real-time data obtained from the sensors to determine the heat strain the worker is experiencing. The algorithm then output the data on a scale of 0-10 heat strain based on resting/current values of metabolic strain and core temperature (see Moran D S et al., Am J Physiol 275 (1 Pt 2): R129-34, 1998, which is incorporated herein by reference). The algorithm calculates a metric that represents feedback of the core temperature or an extrapolation of core temperature. Based on the physiological strain index of the user, the thermoelectric elements (6) described below will be selectively and systematically controlled to provide a cooling or heating affect to certain muscle groups and/or organs to maintain the body temperature of the user in a safe zone, while maintaining the overall comfort level of the user. One of the problems with previous attempts to provide a cooling or warming garment is that the cooling or heating effect is usually an “all-or-nothing” effect. In other words, the cooling or warming zones in the garment are either all on, or all off, depending on whether the user is warm or cold. This is an inefficient way for cooling or warming the user, as the power required to activate all warming or cooling zones will be more than a selective activation. Moreover, generally cooling or warming a garment will be less comfortable for the user than systematically cooling or warming a zone, which could be a target muscle group or organ, that is generating more heat or that is cold.

The controller (4) can systematically control the thermoelectric elements (6) by varying the current and voltage sent to the thermoelectric elements (6). The thermoelectric elements (6) are distributed throughout the garment in a network forming different zones or locales that correspond with a particular muscle group, organ, tissue or pulse point on the human body. Pulse points are points on the human body where a pulse can be detected because the blood vessels are close to the surface of the skin. Applying a cooling agent, such a cloth soaked in cold water, has been shown to quickly and effectively bring down the internal temperature of the human body. Pulse points are found at the wrists, neck, insides of the elbows and knees, tops of the feet, insides of the ankles, and inner thighs. Therefore, thermoelectric elements (6) provided in zones encompassing these pulse points can be activated to quickly bring down the internal body temperature of the user.

The controller (4) can be provided as part of the garment (3), for example, contained within one of the layers of the garment. Alternatively, the controller can be attached to the garment (3) by way of a pocket or holder on either the inner or outer layer of the garment. In another embodiment, the controller (4) is attached to a belt or arm/leg-band worn by the user. In embodiments where the controller (4) also contains an environmental sensor(s), mounting the controller (4) on the outside of the garment, or providing the controller on a belt, arm/leg-band would allow such data to be obtained. If the battery (5) is housed in the controller unit (4), then it would be beneficial for the unit (4) or portion thereof to be accessible for charging or removal of the battery (5).

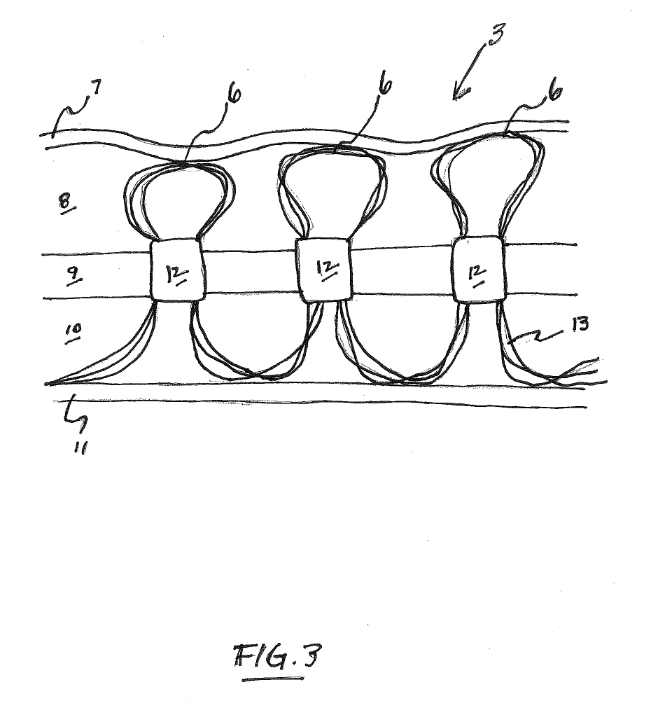

As shown in FIG. 3, the garment (3) is a multi-layered garment. In one embodiment, the garment has five layers: an outer layer (7); a heat-sinking layer (8); an insulating layer (9); a cooling layer (10); and an inner layer (11). The multi-layered garment (3) can be provided as a quilted garment or can be provided as a sectioned or continuous garment, i.e. without quilting, depending on the application. The outer layer (7) is exposed to the environment and is typically made from a waterproof or moisture resistance material, which can also be thermally conductive and electrically insulated. However, in some applications, the outer layer (7) may be provided using a plastic or coated aluminum material. The heat-sinking layer (8) houses the hot side of the thermoelectric elements (6). The insulating layer (9) is made from an electrically and thermally insulated material, such as, but not limited to, a hydrophobic nanofoam. One particularly useful hydrophobic nanofoam is aerogel. In another non-limiting embodiment, the insulating layer (9) is made of plastic and is molded in such a way that when a fan is used, the air will be directed over the hot side of the thermoelectric elements (6). The thermoelectric junctions (12) of the thermoelectric junctions (6) are provided in the insulating layer (9). The cooling layer (10) houses the cold side of the thermoelectric elements (6). The inner layer (11) is closest to the body of the user and is made of a material that is thermally conductive and electrically insulated. It is also preferred that the material is capable of laterally dispersing heat or cold from the thermoelectric elements (6). Moreover, the material is preferably moisture wicking and capable of transferring heat away from the user. Examples of thermally conductive materials include, but are not limited to, plastics, graphite, thermally conductive textiles and fabrics. Insulating fabrics can include, but are not limited to, polarfleece, aramids/para-aramids/meta-aramids or other traditionally insulating materials.

In another embodiment, where the garment (3) provides a heating function, the heat-sinking layer (8) and the cooling layer (10) are reversed, either in orientation or in the flow of electricity, so that the hot sides of the thermoelectric elements (6) are closest to the user and the cold sides are directed towards the environment.

In a further embodiment, the garment (3) is provided to allow both heating and cooling functions. In this case, the heating-sinking layer (8) and the cooling layer (10) contain both the hot and cold sides of the thermoelectric elements (6).

Although the garment (3) has been described as having distinct layers, it should be understood that two or more of these layers can be combined into a single layer having the features of the outer layer (7); heat-sinking layer (8); insulating layer (9); cooling layer (10); and the inner layer (11) described above.

Distributed throughout the garment (3) are thermoelectric elements (6), which provide cooling and heating functions. The thermoelectric elements (6) make use of the Peltier effect to provide a cooling effect on one side of the thermoelectric junction (12) with the other side of the junction (12) providing a heating effect. The thermoelectric elements are preferably provided as a ribbon that transverses the heating-sinking layer (8), the insulating layer (9) and the cooling layer (10). The thermoelectric ribbon (13) is expanded and spread out at each thermoelectric element (6) in both the heating-sinking layer (8) and the cooling layer (10). In another embodiment, the thermoelectric elements (6) are provided as separate, but interlinked modules. In some applications, providing the thermoelectric elements (6) as interlinked modules improves the regional cooling properties of the garment. To prevent short circuiting of the system, each thermoelectric element (6) should be electrically isolated from one another.

The thermoelectric ribbon (13) is typically made from braided, meshed, stranded or woven wire, which is capable of being expanded and spread out in the heat-sinking layer (8) and the cooling layer (10). Such thermoelectric ribbons (13) are described in CA2810857, which is incorporated herein by reference, and those commercially available through Tempronics Inc.

As described above, the thermoelectric elements (6) are connected to the controller unit (4) and controlled by varying current and voltage sent to the thermoelectric elements. The density of the thermoelectric elements (6) in the garment (3) will differ as a function of anatomy, with greater density of elements (6) being concentrated on muscle groups and pulse points.

In an alternate embodiment, fans (not shown) can be provided within the multi-layered garment (3) or on the surface thereof to dissipate heat from the heat-sinking layer (8) and/or outer layer (7).

The systems described above are useful in a variety of different environments and scenarios. For example, workers in deep underground mines are often faced with working in an environment where the ambient temperatures are at or above 30° C. and humidity levels can be in excess of 60%. Therefore, garments controlled by the system described above can allow the worker to work longer in this harsh environment before having to take a break. Moreover, the potential for the worker to suffer heat exhaustion, or other heat-related ailments, will be decreased. Other workers that could benefit from the system described herein include: firefighters, athletes, workers wearing hazmat suits, bomb disposal or military Personal Protective Equipment (PPE), surgeons and construction workers. On the other hand, workers that experience frigid temperatures, such as construction workers in northern climates, may benefit from the system described herein where the garment provides a heating function.

It will be understood that numerous modifications thereto will appear to those skilled in the art. Accordingly, the above description and accompanying drawings should be taken as illustrative of the invention and not in a limiting sense. It will further be understood that it is intended to cover any variations, uses, or adaptations of the invention following, in general, the principles of the invention and including such departures from the present disclosure as come within known or customary practice within the art to which the invention pertains and as may be applied to the essential features herein before set forth, and as follows in the scope of the appended claims.

1-25. (canceled)

26. A system for regulating body temperature of a subject, said system comprising:
a multi-layered garment comprising a plurality of thermoelectric elements integrated throughout the multi-layered garment, the multi-layered garment comprising an outer layer, a heat-sinking layer, an insulating layer, a heating or cooling layer, and an inner layer, wherein the thermo-electric elements transverse the heat-sinking, insulating and heating or cooling layers;
one or more sensors attached to the outside of the multi-layered garment for monitoring environmental conditions and one or more sensors positioned on or near the subject for monitoring physiological parameters;
a controller for receiving input from the one or more sensors and connected to the plurality of thermoelectric elements to systemically control the thermoelectric elements based on the environmental and physiological parameters; and
a battery for providing power to the one or more sensors, the thermoelectric elements, the controller or a combination of any of these.

27. The system according to claim 26, wherein the environmental conditions are ambient temperature, humidity, barometric pressure, air velocity or any combination of these.

28. The system according to claim 26, wherein the physiological parameters are body temperature, heart rate, breathing rate, muscle activity, skin temperature, heat flux or a combination of these.

29. The system according to claim 26, wherein the outer layer is thermally conductive and electrically insulated.

30. The system according to claim 26, wherein the outer layer is waterproof.

31. The system according to claim 26, wherein the insulating layer is electrically and thermally insulated.

32. The system according to claim 26, wherein the inner layer is thermally conductive and electrically insulated.

33. The system according to claim 32, wherein the inner layer transfers heat away from the subject.

34. The system according to claim 26, wherein the thermoelectric elements are controlled by pulse width modulation or varying the current, voltage, or both, supplied to the thermoelectric elements.

35. The system according to claim 26, wherein the garment is a vest, jacket, trousers, jumpsuit, hat, helmet, or any combination of these.

36. The system according to claim 26, further comprising fans for cooling the heat-sinking layer, insulating layer, outer layer, or any combination of these.

37. The system according to claim 26, wherein the battery is mounted on the garment.

38. The system according to claim 26, wherein one of the sensors for monitoring physiological parameters is for mounting intraaurally to measure body temperature and heart rate.

39. The system according to claim 26, wherein one or more of the sensors for monitoring physiological parameters is for mounting on the skin of the subject.

40. The system according to claim 26, wherein the battery is a portable battery.

41. The system according to claim 26, wherein one or more of the sensors for monitoring physiological parameters is a non-invasive physiological sensor.

42. A method for regulating body temperature of a subject, comprising the steps of:
obtaining environmental conditions and physiological parameters from the one or more sensors attached to the outside of the multi-layer garment according to claim 2 and the one or more sensors positioned on or near the subject;
processing said environmental conditions and physiological parameters in the controller; and
systematically controlling the plurality of thermoelectric elements integrated throughout the multi-layer garment worn by the subject based on said environmental conditions and physiological parameters.
